(19) United States
(12) Patent Application Publication
(10) Pub. No.: US 2009/0198321 A1
(43) Pub. Date: Aug. 6, 2009

(54) Drug-Coated Medical Devices for Differential Drug Release

(75) Inventors: Derek Sutermeister, Eden Prairie, MN (US); Benjamin Arcand, Minneapolis, MN (US); Raed Rizq, Fridley, MN (US); Adam Wentland, Becker, MN (US); Jay Rassat, Buffalo, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/363,011

(22) Filed: Jan. 30, 2009

Related U.S. Application Data
(60) Provisional application No. 61/025,576, filed on Feb. 1, 2008.

(51) Int. Cl. A61F 2/06

(52) U.S. Cl. 623/1.42; 623/1.46

(57) ABSTRACT

The embodiments described herein relate generally to medical devices that are useful for delivering one or more therapeutic agents to a body tissue of a subject, and methods for making and using such devices. In particular, the embodiments relate to a medical device such as a stent having a sidewall structure comprising a plurality of struts, in which one or more therapeutic agents are released from different surfaces of the struts at different release profiles. More particularly, the embodiments relate to a stent having a sidewall structure comprising a plurality of struts, in which the two side surfaces of each strut are coated with a coating composition that is different from the coating composition used to coat the outer surface and/or inner surface of the strut.

### CROSS-REFERENCE TO RELATED APPLICATIONS

This application claims priority from Provisional Application No. 61/025,576, filed Feb. 1, 2008, the contents of which is hereby incorporated by reference

### 1. FIELD OF THE INVENTION

Disclosed herein are medical devices that are useful for delivering one or more therapeutic agents to a body tissue of a subject, and methods for making and using such medical devices. In particular, the medical device, such as a stent, has a sidewall structure comprising a plurality of struts, in which one or more therapeutic agents are differentially released from different surfaces of the struts.

### 2. BACKGROUND OF THE INVENTION

Cardiovascular disease is a leading cause of death in the developed world. Patients having such disease usually have narrowing or closing (stenosis) in one or more arteries. The use of medical devices, such as stents, in the treatment of cardiovascular disease is well known. Stents are typically delivered in a contracted state to the treatment area within a lumen, where they are then expanded. The use of expandable stents, e.g., balloon-expandable and self-expanding stents, however, may cause additional trauma to a body lumen upon deployment of the stent, and may lead to re-narrowing (i.e., restenosis) of the body lumen.

Recently, various types of drug-coated stents have been used for the localized delivery of therapeutic agents to the body lumen wall to reduce the likelihood of restenosis. Many drug-coated stents are designed to have identical drug release profiles from all surfaces, i.e., the outer surface (abluminal surface), the inner surface (abluminal/luminal surface), and the side surfaces connecting the outer and inner surfaces. These types of stents pose certain limitations. For example, it has been shown that endothelial cell growth after stent implantation does not take place along all surfaces of the stent struts simultaneously.

Accordingly, it is desirable to have a medical device (e.g., a stent) that is capable of differentially releasing one or more therapeutic agents from different surfaces of the medical device.

### 3. SUMMARY OF THE INVENTION

The medical devices described herein include an insertable or implantable medical device such as a stent, having a sidewall structure comprising a plurality of struts, in which one or more therapeutic agents are differentially released from different surfaces of the struts.

In one embodiment, the coated medical device comprises a tubular sidewall that comprises a plurality of struts, each having an outer surface that is adapted for exposure to a body lumen, an inner surface opposite the outer surface, and at least one side surface adjacent to the outer surface and the inner surface and connecting the outer surface and the inner surface. For each strut, the outer surface of at least one strut has at least a portion that is coated by a first coating composition by a first coating process, and each of the side surfaces of said at least one strut has at least a portion that is coated by a second coating composition by a second coating process, wherein the first coating composition comprises a first therapeutic agent and a first polymer, and the second coating composition comprises a second therapeutic agent and a second polymer. These two coating compositions differ in at least one aspect, e.g., quantity or type of constituents (e.g., therapeutic agent, polymer, and solvent) and/or formulation of the compositions (e.g., method of preparation), and/or application of the composition, such that the first therapeutic agent is released with a release profile (e.g., release rate, time of release, (e.g., starting/ending time of release or duration of release), and/or total amount released) which is different than the release profile of the second therapeutic agent. In one embodiment, the tubular sidewall forms a stent.

In some embodiments, the inner surface and the side surfaces of said at least one strut are not coated by the first coating composition, and the outer surface and the inner surface of said at least one strut are not coated by the second coating composition.

In some embodiments, the inner surface of said at least one strut has at least a portion that is coated by the same coating composition covering the outer surface of said at least one strut, and by the same coating process with which said same coating composition is coated onto the outer surface.

In some embodiments, the inner surface of said at least one strut has at least a portion that is coated by a third coating composition by a third coating process, wherein the third coating composition comprises a third therapeutic agent and third polymer, and is different from the first coating composition covering the outer surface of said at least one strut and the second coating composition covering the side surfaces of said at least one strut in at least one aspect, such that the third therapeutic agent is released with a release profile which is different than the release profile of the first therapeutic agent and the release profile of the second therapeutic agent.

In another embodiment, for each strut, the inner surface of at least one strut has at least a portion that is coated by a first coating composition by a first coating process, and each of the side surfaces of said at least one strut has at least a portion that is coated by a second coating composition by a second coating process, wherein the first coating composition comprises a first therapeutic agent and a first polymer, and the second coating composition comprises a second therapeutic agent and a second polymer. These two coating compositions differ in at least one aspect such that the first therapeutic agent is released with a release profile which is different than the release profile of the second therapeutic agent.

In some embodiments, the outer surface and the side surfaces of said at least one strut are not coated by the first coating composition, and the outer surface and the inner surface of said at least one strut are not coated by the second coating composition.

In some embodiments, the therapeutic agents in the different coating compositions are the same. In other embodiments, the therapeutic agents in the different coating compositions are different. In certain embodiments, one or more of the coating compositions do not comprise any therapeutic agent.

In some embodiments, the polymers in the different coating compositions are the same. In other embodiments, the polymers in the different coating compositions are different.

In some embodiments, one or more of the polymers in the different coating compositions are biocompatible.

In some embodiments, one or more of the coating compositions are biodegradable.

In some embodiments, the coating compositions are being coated onto different surfaces of the strut by the same process. In other embodiments, the coating compositions are being coated onto different surfaces of the strut by different processes.

### 4. FIGURES

The embodiments described herein can be explained, for example, with reference to the following drawings:

FIG. 1A depicts a perspective view of an implantable intravascular stent, having a sidewall comprising a plurality of struts with an outer surface, an inner surface, and side surfaces.

FIG. 1B shows a portion of a strut that can be part of a stent, the strut has an outer surface, an inner surface, and two side surfaces, and the cross-section of the strut is rectangular.

FIG. 1C shows a portion of a strut that can be part of a stent, the strut has an outer surface, an inner surface, and two side surfaces, and the cross-section of the strut is circular.

FIG. 2A shows a cross-sectional view of a strut of a stent that has a first coating composition disposed on its outer surface and a second coating composition disposed on its side surfaces.

FIG. 2B shows a cross-sectional view of a strut of a stent that has a first coating composition disposed on its outer surface and inner surface and a second coating composition disposed on its side surfaces.

FIG. 2C shows a cross-sectional view of a strut of a stent that has a first coating composition disposed on its outer surface, a second coating composition disposed on its side surfaces, and a third coating composition disposed on its inner surface.

FIG. 2D shows a cross-sectional view of a strut of a stent that has a first coating composition disposed on its inner surface and a second coating composition disposed on its side surfaces.

### 5. DETAILED DESCRIPTION OF THE INVENTION

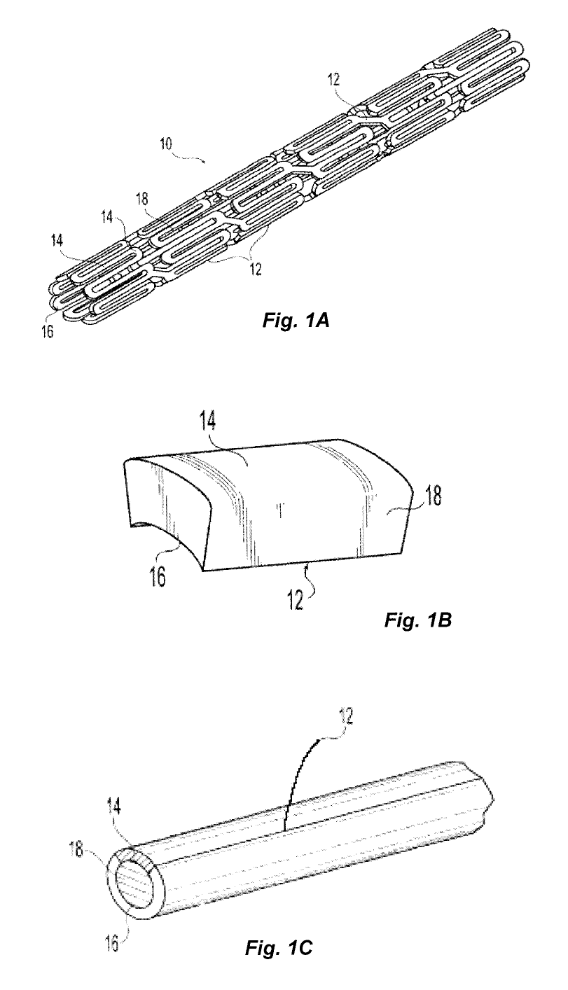

As shown in FIG. 1A, a medical device, such as an implantable stent, comprises a sidewall structure 10 comprising a plurality of struts 12. The struts 12 may be arranged in any suitable configuration. The struts 12 do not all have to have the same shape or geometric configuration. For example, the cross-sectional view of the strut 12 can be a square/rectangle as depicted in FIG. 1B, or a circle/ellipse as depicted in FIG. 1C. Generally, each strut 12 has an outer (i.e., abluminal) surface 14, an inner (i.e., abluminal/luminal) surface 16 opposite the outer surface 14, and at least one side surface 18. The outer surface 14 of the strut 12 is the surface that comes in direct contact with the body lumen wall when the stent is implanted. The outer surface 14 need not include only one flat surface or facet. Instead, it can be rounded, such as in the case of a wire strut 12, have a number of facets, or have a number of microstructures (e.g., micro-needle, micro-pore, micro-cylinder, micro-cone, micro-pyramid, micro-tube, micro-parallelepiped, micro-prism, micro-hemisphere, teeth, rib, ridge, ratchet, hinge, zipper, zip-tie like structure, etc.). The inner surface 16 of the strut 12 is the surface that is opposite the outer surface 14 and generally faces the interior of the body lumen. The two side surfaces 18 of the strut 12 are the surfaces that are adjacent to the outer surface 14 and/or inner surface 16. The side surfaces 18 connect the outer surface 14 and inner surface 16. Like the outer surface 14, the inner surface 16 and side surfaces 18 can be rounded and/or have a number of facets or microstructures.

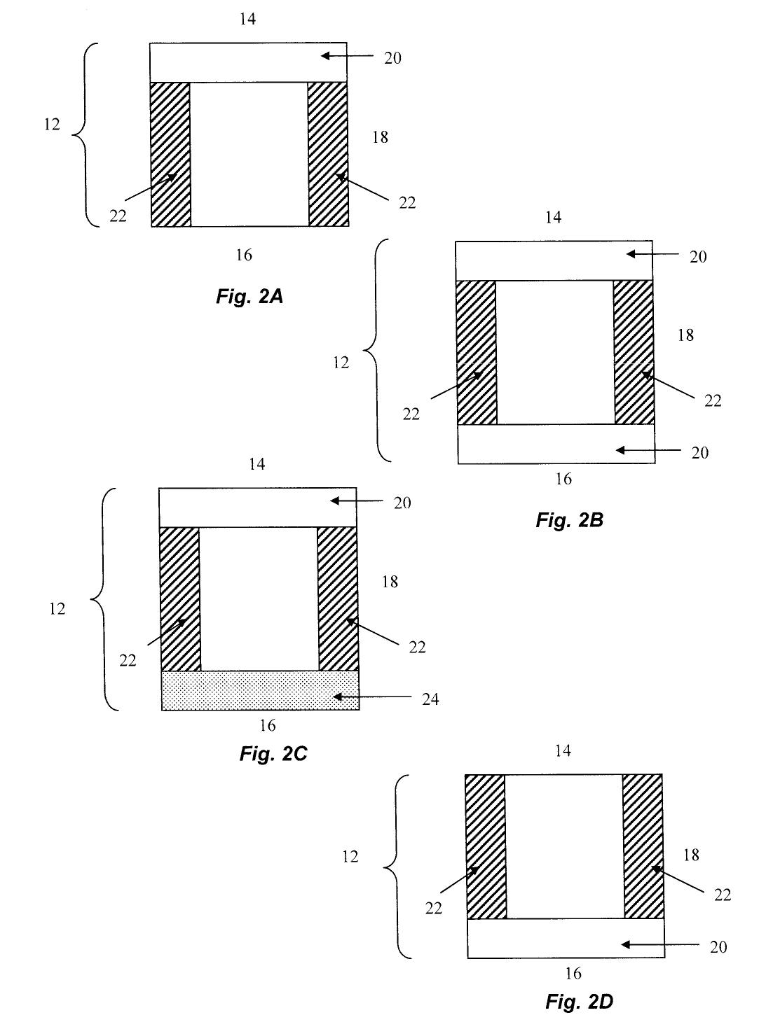

FIGS. 2A-2D show cross sectional views of a strut in different exemplary, non-limiting embodiments of the device. As shown in FIG. 2A, a first coating composition 20 comprising a first therapeutic agent and a first polymer is disposed on at least a portion of the outer surface 14 of the strut 12, and a second coating composition 22 comprising a second therapeutic agent and a second polymer is disposed on at least a portion of the side surfaces 18 of the strut 12. The inner surface 16 of the strut 12 is free of any coating composition.

In an alternative embodiment shown in FIG. 2B, the first coating composition 20 is disposed on at least a portion of the outer surface 14 of the strut 12 and at least a portion of the inner surface 16 of the strut 12, and the second coating composition 22 is disposed on at least a portion of the side surfaces 18 of the strut 12.

In an alternative embodiment shown in FIG. 2C, the first coating composition 20 is disposed on at least a portion of the outer surface 14 of the strut 12, and the second coating composition 22 is disposed on at least a portion of the side surfaces 18 of the strut 12. In addition, a third coating composition 24 comprising a third therapeutic agent and a third polymer is disposed on at least a portion of the inner surface 16 of the strut 12.

In an alternative embodiment shown in FIG. 2D, the first coating composition 20 is disposed on at least a portion of the inner surface 16 of the strut 12, and the second coating composition 22 is disposed on at least a portion of the side surfaces 18 of the stent 12.

Although not shown in the figures, the two side surfaces 18 of the strut 12 may each be coated with a different coating composition. In addition, the outer surface 14, the inner surface 16, and each of the side surfaces 18 of the strut 12 may individually comprise two or more different coating compositions. Furthermore, one or more of the coating compositions on various surfaces of the strut 12 may comprise no therapeutic agent, yet is capable of exerting an effect in reducing or preventing stenosis or restenosis.

The medical devices are discussed in more detail in Section 5.1 infra. Methods of preparing and using the medical devices are discussed in Sections 5.2 and 5.3, respectively, infra. For clarity of disclosure, and not by way of limitation, the detailed description of the embodiments is divided into the subsections which follow.

### 5.1 Drug-Coated Medical Devices

### 5.1.1 Types of Medical Devices

The medical devices can be implanted or inserted into the body of a subject. Suitable medical devices include, but are not limited to, those that have a tubular or cylindrical like portion. For example, the tubular portion of the medical device need not be completely cylindrical. The cross-section of the tubular portion can be any shape, such as rectangle, a triangle, etc., not just a circle.

In addition, the tubular portion of the medical device may be a sidewall that may comprise a plurality of struts defining a plurality of openings. The struts may be arranged in any suitable configuration. Also, the struts do not all have to have the same shape or geometric configuration. When the medical device is a stent comprising a plurality of struts, the surface is located on the struts. Each individual strut has an outer surface adapted for exposure to the body tissue of the patient, an inner surface, and at least one side surface adjacent to the outer surface and the inner surface and connecting the outer surface and inner surface.

Medical devices that are particularly suitable include any kind of stent for medical purposes which is known to the skilled artisan. For example, the medial devices can be, but are not limited to, e.g., stents (e.g., bifurcated stents), surgical staples, catheters (e.g., balloon catheters, central venous catheters, and arterial catheters), guidewires, cannulas, cardiac pacemaker leads or lead tips. Preferably, the stents are intravascular stents that are designed for permanent implantation in a blood vessel of a patient. In certain embodiments, the stent comprises an open lattice sidewall stent structure, such as coronary stent. Other suitable stents include, for example, vascular stents such as self-expanding stents and balloon-expandable stents. Examples of self-expanding stents are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. When the coatings are applied to a stent having openings in the stent sidewall structure, in certain embodiments, it is preferable that the coatings conform to the surface of the stent so that the openings in the sidewall stent structure are preserved, e.g., the openings are not entirely or partially occluded with coating material.

The framework of suitable stents may be formed through various methods as known in the art. The framework may be welded, molded, laser cut, electro-formed, or consist of filaments or fibers which are wound or braided together in order to form a continuous structure.

Suitable substrates of the medical device (e.g., stents) may be fabricated from a metallic material, ceramic material, polymeric material, or a combination thereof (see Sections 5.1.1.1 to 5.1.1.3 infra.). Preferably, the materials are biocompatible. The material may be porous or non-porous, and the porous structural elements can be microporous or nanoporous.

### 5.1.1.1 Metallic Materials for Device Formation

In certain embodiments, the medical device comprises a substrate which is metallic. Suitable metallic materials useful for making the substrate include, but are not limited to, metals and alloys based on titanium (e.g., nitinol, nickel titanium alloys, and thermo memory alloy materials), stainless steel, gold, platinum, iridium, molybdenum, niobium, palladium, chromium, tantalum, nickel chrome, or certain cobalt alloys including cobalt chromium nickel alloys such as Elgiloy® and Phynox®, or a combination thereof. Other metallic materials that can be used to make the medical device include clad composite filaments, such as those disclosed in WO 94/16646.

Preferably, the metal region comprises a radiopaque material. Including a radiopaque material may be desired so that the medical device is visible under X-ray or fluoroscopy. Suitable materials that are radiopaque include, but are not limited to, gold, tantalum, platinum, bismuth, iridium, zirconium, iodine, titanium, barium, silver, tin, alloys of these metals, or a combination thereof.

Furthermore, although the medical devices can be practiced by using a single type of metal to form the substrate, various combinations of metals can also be employed. The appropriate mixture of metals can be coordinated to produce desired effects when incorporated into a substrate.

### 5.1.1.2 Ceramic Materials for Device Formation

In certain embodiments, the medical device comprises a substrate which is ceramic. Suitable ceramic materials used for making the substrate include, but are not limited to, oxides, carbides, or nitrides of the transition elements such as titanium oxides, platinum oxide, tantalum oxide, hafnium oxides, iridium oxides, chromium oxides, niobium oxide, tungsten oxide, rhodium oxide, aluminum oxides, zirconium oxides, or a combination thereof. Silicon based materials, such as silica, may also be used.

Furthermore, although the medical devices can be practiced by using a single type of ceramic to form the substrate, various combinations of ceramics can also be employed. The appropriate mixture of ceramics can be coordinated to produce desired effects when incorporated into a substrate.

### 5.1.1.3 Polymeric Materials for Device Formation

In certain embodiments, the medical device comprises a substrate which is polymeric. Suitable polymeric materials for making the substrate should be ones that are biocompatible, particularly during insertion or implantation of the device into the body and avoid irritation to body tissue. Examples of such polymers include, but are not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, poly(lactic-co-glycolic acid), polylactic acid-polyethylene oxide copolymers, styrene-isobutylene-styrene, and styrene and isobutylene copolymers.

When the polymer is being used to form a part of the medical device, such as a stent which undergoes mechanical challenges, e.g., expansion and contraction, the polymers are preferably selected from elastomeric polymers such as silicones (e.g., polysiloxanes and substituted polysiloxanes), polyurethanes, thermoplastic elastomers, ethylene vinyl acetate copolymers, polyolefin elastomers, and EPD (ethylene-propylene-diene) rubbers. The polymer is selected to allow the coating to better adhere to the surface of the strut when the stent is subjected to forces or stress.

Furthermore, although the medical devices can be practiced by using a single type of polymer to form the substrate, various combinations of polymers can also be employed. The appropriate mixture of polymers can be coordinated to produce desired effects when incorporated into a substrate.

### 5.1.2 Coating Compositions

The coating compositions are different if they are different in at least one aspect. For instance, the coating compositions may differ in the quantity and/or type of constituent(s) (e.g., therapeutic agent, polymer, and solvent), and/or formulation of the compositions (e.g., method of preparation), and/or application of the composition. For example, a medical device can comprise a first coating composition comprising a first therapeutic agent and a first polymer, and being coated by a first coating process to one surface of a strut of the medical device, and a second coating composition comprising a second therapeutic agent and a second polymer, and being coated by a second coating process to another surface of the strut, and the coating compositions may differ according to one of the following ways: (1) the first and the second therapeutic agents being the same, the first and second polymers being the same, and the first and second coating processes being different; (2) the first and the second therapeutic agents being the same, the first and the second polymers being different, and the first and the second coating processes being the same; (3) the first and the second therapeutic agents being different, the first and the second polymers being the same, and the first and the second coating processes being the same; (4) the first and the second therapeutic agents being the same, the first and the second polymers being different, and the first and the second coating processes being different; (5) the first and the second therapeutic agents being different, the first and the second polymers being the same, and the first and the second coating processes being different; (6) the first and the second therapeutic agents being different, the first and the second polymers being different, and the first and the second coating processes being the same; and (7) the first and the second therapeutic agents being different, the first and the second polymers being different, and the first and the second coating processes being different.

### 5.1.2.1 Therapeutic Agents

The term “therapeutic agent” as used herein encompasses drugs, genetic materials, biological materials, and their analogs or derivatives thereof, and can be used interchangeably with “biologically active material”. The term “genetic materials” means DNA or RNA, including, without limitation, DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other suitable therapeutic agents include:
anti-thrombogenic agents such as heparin, heparin derivatives, streptokinase, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins, e.g. Abraxane™;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, daunomycin, mitocycin;
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds;
macrolides such as sirolimus (rapamycin) or everolimus; and
AGE-breakers including alagebrium chloride (ALT-711)

Other therapeutic agents include nitroglycerin, nitrous oxides, nitric oxides, antibiotics, aspirins, digitalis, estrogen, estradiol and glycosides. Preferred therapeutic agents include anti-proliferative drugs such as steroids, vitamins, and restenosis-inhibiting agents. Preferred restonosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the medical devices include 2′-succinyl-taxol, 2′-succinyl-taxol triethanolamine, 2′-glutaryl-taxol, 2′glutaryl-taxol triethanolamine salt, 2′-O-ester with N-(dimethylaminoethyl)glutamine, and 2′-O-ester with N-(dimethylaminoethyl)glutamide hydrochloride salt.

Other preferred therapeutic agents include tacrolimus; halafuginone; inhibitors of HSP90 heart shock proteins such as geldanamysin; microtubule stabilizing agents such as epothilone D; phosphodiesterase inhibitors such as cliostazole; Barkct inhibitors; phospholamban inhibitors; and Serca 2 gene/proteins. In yet another preferred embodiment, the therapeutic agent is an antibiotic such as erythromycin, amphotericin, rapamycin, adriamycin, etc.

In preferred embodiments, the therapeutic agent comprises daunomycin, mitocycin, dexamethasone, everolimus, tacrolimus, zotarolimus, heparin, aspirin, warfarin, ticlopidine, salsalate, diflunisal, ibuprofen, ketoprofen, nabumetone, prioxican, naproxen, diclofenac, indomethacin, sulindac, tolmetic, etodolac, ketorolac, oxaprozin, celcoxib, alagebrium chloride or a combination thereof.

In another preferred embodiment, the therapeutic agent can be anti-TNF agents, antiplatelet agents, thrombogenics, viral growth agents, fatty acids (e.g., omega-3, omega-6), live cultures, zinc or other inorganic compounds or elements, and fiber or other organic compounds.

In another preferred embodiment, the therapeutic agent comprises an antiproliferative agent, an anticontraction agent, an antimigratory agent, an anti-hyperactivity agent, an anti-thrombogenic agent, or a combination thereof. In one embodiment, the antiproliferative agent is paclitaxel, an analogue or derivative thereof, or a combination thereof.

The therapeutic agents can be synthesized by methods well known to one skilled in the art. Alternatively, the therapeutic agents can be purchased from chemical and pharmaceutical companies.

In some embodiments of the medical devices, the physiochemical properties of a therapeutic agent, e.g., particle size, shape, mass or nature (e.g., hydrophobic versus hydrophilic, anionic versus cationic, etc.), can be modified to vary the rate that said therapeutic agent is released from the surfaces of a strut of a stent.

Methods suitable for applying and/or incorporating therapeutic agents to the devices preferably do not alter or adversely impact the therapeutic properties of the therapeutic agent.

### 5.1.2.2 Polymers

Polymers may be used to carry the therapeutic agent and coated onto the medical device. For example, when a hydrophilic therapeutic agent is used, a hydrophilic polymer having a greater affinity for the therapeutic agent than another material that is less hydrophilic may be used to carry the agent. When a hydrophobic therapeutic agent is used, a hydrophobic polymer having a greater affinity for the therapeutic agent may be used to carry the agent.

Examples of suitable hydrophobic polymers or monomers include, but are not limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluorpropyl methacrylate copolymers; halogenated hydrocarbon polymers, such as poly(chlorotrifluoroethylene), chlorotrifluorethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyle methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrohexafluoroisopropyl methacrylate), poly(1,1-dihydrotetrafluoropropyl methacrylate), poly(1-hydrohexafluoroisopropyl methacrylate), and poly(t-nonafluorobutyl methacrylate); polyesters, such as poly(ethylene terephthalate) and poly(butylene terephthalate); condensation type polymers such as polyurethanes and siloxane-urethane copolymers; polyorganosiloxanes, i.e., polymeric materials characterized by repeating siloxane groups, represented by Ra SiO 4-a/2, where R is a monovalent substituted or unsubstituted hydrocarbon radical and the value of a is 1 or 2; and naturally occurring hydrophobic polymers such as rubber.

Examples of suitable hydrophilic polymers or monomers include, but are not limited to, (meth)acrylic acid, or alkaline metal or ammonium salts thereof, (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl(meth)acrylate and 2-hydroxypropyl(meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO₃). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

Other suitable polymers include without limitation: polyurethanes, silicones (e.g., polysiloxanes and substituted polysiloxanes), and polyesters, styrene-isobutylene-styrene, styrene-isobutylene-copolymers. Other polymers which can be used include ones that can be dissolved and cured or polymerized on the medical device or polymers having relatively low melting points that can be blended with therapeutic agents. Additional suitable polymers include, but are not limited to, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, polyether block amides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, poly(lactic-co-glycolic acid), polylactic acid-polyethylene oxide copolymers, EPD (ethylene-propylene-diene) rubbers, fluoropolymers, fluorosilicones, polyethylene glycol, polysaccharides, phospholipids, and combinations of the foregoing.

In a preferred embodiment, the polymer can be electro-active polymers (ionic or gel), shape memory polymer, polyanhydride, ethylene vinyl alcohol polymers, and poly(d-lactic acid).

Furthermore, the same type of polymer can have different properties (e.g., orientation, surface affinity) by being treated with different solvent systems, and, as a result, can affect the release rate of one or more therapeutic agent(s) from a coating composition comprising said polymer. For example, solvent evaporation can change the surface characteristics of the polymer.

Although the medical devices can be practiced by using a single type of polymer to form the substrate, various combinations of polymers can also be employed. The appropriate mixture of polymers can be coordinated to produce desired effects when incorporated into a coating composition.

### 5.1.2.3 Release Profiles of Therapeutic Agent

The release profile of a therapeutic agent from different surfaces of a medical device may vary. In one aspect, one or more therapeutic agent(s) are released from different surfaces of a medical device at different release rates (i.e., amount released per time unit), different times of release (e.g., starting/ending time of release or duration of release), and/or different amounts released. For example, in one embodiment, of the medical device, a therapeutic agent is released from a side surface of a strut of the medical device at a different (i.e., higher or lower) rate, a different (i.e., earlier or later) starting/ending time, a different (i.e., longer or shorter) duration, and/or a different (i.e., greater or less) amount than the rate, starting time, duration, and/or amount at which said therapeutic agent and/or other therapeutic agent(s) are being released from an outer surface and an inner surface of the strut.

In some embodiments, the release rate, time of release, and/or total amount released at which a therapeutic agent is released from one surface (e.g., outer surface, inner surface, and/or side surface(s)) of a strut of a medical device is about ten times, nine times, eight times, seven times, six times, five times, four times, three times, or two times higher/lower than the release rate, longer/shorter and/or earlier/later than the time of release, and/or greater/less than the total amount released at which the same therapeutic agent and/or different therapeutic agent(s) are being released from another surface of the strut. For example, there can be time delays between the release of therapeutic agents from different surfaces of the medical device.

The release profiles of the therapeutic agent(s) from different surfaces of the medical device may be adjusted based on a variety of factors, such as the therapeutic agents being used and the desired therapeutic effects to be achieved. For example, the physiochemical properties of (1) the therapeutic agent(s) (e.g., particle size, shape, mass, or nature) and the polymer(s) of the coating compositions, (2) the solvent(s) used to prepare the coating compositions, (3) the process with which the coating compositions are coated onto the surfaces of the medical device, or a combination thereof, can be modified based on knowledge in the art.

The release profile of a therapeutic agent can be measured by any method known by one skilled in the art. For example, the release rate may be measured by placing the coated medical device in a solution containing aqueous and/or organic solvent(s) and sampling the amount of the therapeutic agent(s) released from different surfaces of the medical device into the solution using an analytic instrument (e.g., high-performance liquid chromatography) at different time intervals. The amount of therapeutic agent being measured can be the total amount of the therapeutic agent released, the amount of therapeutic agent released as a percentage of the total amount deposited on the coated medical device, or the percentage amount of therapeutic agent released per surface area of the coated medical device. Furthermore, different sized particles of the same therapeutic agent may be differentially released, and the release profile(s) of which can be measured by staining the drug particles.

### 5.2 Methods of Making the Medical Devices

### 5.2.1 Methods of Making a Coating Composition

The coating compositions can be prepared by any method known by one skilled in the art. For example, the coating composition can be prepared by dissolving or suspending a polymer/polymers and/or a therapeutic agent/therapeutic agents in a solvent/solvents. Solvents that may be used to prepare coating compositions include ones which can dissolve or suspend the polymer and/or therapeutic agent in solution. Examples of suitable solvents include, but are not limited to, tetrahydrofuran, methylethylketone, chloroform, toluene, acetone, isooctane, 1,1,1-trichloroethane, dichloromethane, isopropanol, dimethylformamide, ethylacetate, isobutylstyrene, and mixture thereof.

### 5.2.2 Methods of Coating a Medical Device

The coating compositions can be applied to a surface of a medical device by any method known by one skilled in the art. The different surfaces may be coated by the same or different methods. Suitable methods for applying the coating compositions to the medical devices include, but are not limited to, spray coating, painting, roll coating, electrostatic spray deposition, ink jet coating, dip coating, spin coating, air suspension, pan coating, ultrasonic mist spraying, or a combination thereof.

In some embodiments, one or more layers of the same coating composition can be deposited on a surface of a medical device. The same or different coating methods discussed above can be used to apply the one or more layers of coating composition to coat the medical device.

In embodiments where a coating composition is to be applied to fewer than all the surfaces of the struts of a medical device, such as a strut of a stent described above, it is preferable to employ coating methods that selectively apply the coating composition. In one embodiment, the surface that is not to be coated with a particular coating composition may be masked prior to applying the coating composition. By way of example, in the embodiment as shown in FIG. 2A, to avoid having the first coating composition 20 being disposed on the applying the first composition 20 to the outer surface 14. In another embodiment, the masking can be done, for example, by application of a protective wrap. The protective wrap is a material that would protect the coated surface from exposure to the coating applied to the opposing surface. Suitable material for this protective wrap include, for example, PTFE film, dyna-leap, Kapton®, or any other appropriate type of covering or wrapping material.

In an alternative embodiment, a bare stent can be dip coated with a photo resist material such as polymers which are dissolvable or resistant to different solvent systems. In one example, a bare stent can be dip coated with wax or sacrificial masking material. In order to selectively coat particular surfaces of the stent, the wax or sacrificial masking material coating can be ground off in selected surfaces, exposing the chosen surfaces of the stent struts. Subsequently, the stent can be spray coated, dipped, painted, rolled or by other means coated on the exposed surfaces. After the coating is complete, the wax or sacrificial masking material on the remaining surfaces of the stent can be removed.

When the coatings are applied to a stent having openings in the stent sidewall structure, in certain embodiments, it is preferable that the coatings conform to the surface of the stent so that the openings in the sidewall stent structure are preserved, e.g., the openings are not entirely or partially occluded with coating material.

After a coating composition has been applied, it can be cured. Curing is defined as the process of converting the polymeric material into the finished or useful state by the application of heat, vacuum, and/or chemical agents which induce physicochemical changes. The applicable time and temperature for curing are determined by the particular polymer involved and particular therapeutic agent used, if any, as known by one skilled in the art. The coated medical devices may thereafter be subjected to a post-cure process. Also, after the medical device is coated, it preferably should be sterilized by methods of sterilization as known in the art.

### 5.3 Therapeutic Uses

The coated medical devices can be used to treat and/or prevent various diseases, including but not limited to cardiovascular diseases such as stenosis or restenosis, in a subject in need thereof. The coated medical devices can be inserted or implanted into the subject.

The coated medical devices may also be used to treat and/or prevent diseases that may benefit from decreased cell proliferation, contraction, migration, hyperactivity, and/or thrombogenesis, for example, by releasing one or more therapeutic agents useful for inhibiting smooth muscle cell proliferation, contraction, migration, hyperactivity, and/or thrombogenesis.

The medical devices can be used in methods for treating or preventing stenosis or restenosis. In particular, the methods for treating or preventing stenosis or restenosis involve inserting or implanting a coated medical device into a subject. In such applications, the coated medical devices contain a therapeutically effective amount of the therapeutic agent. The therapeutically effective amount of a therapeutic agent for the subject can vary depending on the subject being treated and the therapeutic agent itself. The therapeutically effective amount can also vary with the condition to be treated and the severity of the condition. The dose, and perhaps the dose frequency, can also vary according to the age, gender, body weight, and response of the individual subject.

As used herein, the terms “subject” and “patient” are used interchangeably. The subject can be an animal, preferably a mammal including a non-primate (e.g., a cow, pig, horse, cat, dog, rat, and mouse) and a primate (e.g., a monkey, such as a cynomologous monkey, chimpanzee, and a human), and typically a human.

In one embodiment, the subject can be a subject who had stenosis and/or had undergone a regimen of treatment (e.g., percutaneous transluminal coronary angioplasty (PTCA), also known as balloon angioplasty, and coronary artery bypass graft (CABG) operation).

The medical devices and methods are useful alone or in combination with other treatment modalities. In certain embodiments, the subject can be receiving concurrently other therapies to treat or prevent stenosis or restenosis.

The medical devices and methods are not to be limited in scope by the specific embodiments described which are intended as single illustrations of individual aspects of the medical devices and methods, and functionally equivalent methods and components are contemplated. Indeed, various modifications of the embodiments, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference. Citation or discussion of a reference herein shall not be construed as an admission that such is prior art.

We claim:

1. A coated medical device comprising:
a tubular sidewall, wherein the sidewall comprises a plurality of struts each having an outer surface that is adapted for exposure to a body lumen, an inner surface opposite the outer surface, and at least one side surface adjacent to the outer surface and the inner surface and connecting the outer surface and the inner surface;
wherein the outer surface of at least one strut has at least a portion that is coated by a first coating composition by a first coating process, wherein the first coating composition comprises a first therapeutic agent and first polymer;
wherein the side surface of said at least one strut has at least a portion that is coated by a second coating composition by a second coating process, wherein the second coating composition comprises a second therapeutic agent and second polymer, and wherein the second coating composition is different from the first coating composition;
wherein the first therapeutic agent is released with first release profile and the second therapeutic agent is released with a second release profile which is different than the first release profile.

2. The coated medical device of claim 1, wherein the first release profile and the second release profile differ by release rate, time of release, or total amount released of the therapeutic agents.

3. The coated medical device of claim 1, wherein the tubular sidewall forms a stent.

4. The coated medical device of claim 1, wherein the inner surface and the side surface of said at least one strut are not coated by the first coating composition, and wherein the outer surface and the inner surface of said at least one strut are not coated by the second coating composition.

5. The coated medical device of claim 1, wherein the first therapeutic agent and the second therapeutic agent are the same.

6. The coated medical device of claim 1, wherein the first therapeutic agent and the second therapeutic agent are different.

7. The coated medical device of claim 1, wherein the first polymer and the second polymer are the same.

8. The coated medical device of claim 1, wherein the first polymer and the second polymer are different.

9. The coated medical device of claim 1, wherein the first polymer and the second polymer is biocompatible.

10. The coated medical device of claim 1, wherein the first coating composition or the second coating composition is biodegradable.

11. The coated medical device of claim 1, wherein the first coating process and the second coating process are the same.

12. The coated medical device of claim 1, wherein the first coating process and the second coating process are different.

13. The coated medical device of claim 1, wherein the inner surface of said at least one strut has at least a portion that is coated by the first coating composition by the first coating process.

14. The coated medical device of claim 1, wherein the inner surface of said at least one strut has at least a portion that is coated by a third coating composition by a third coating process, wherein the third coating composition comprises a third therapeutic agent and a third polymer, wherein the third coating composition is different from the first coating composition and is different from the second coating composition, and wherein the third therapeutic agent is released with a third release profile which is different than the first release profile and is different than the second release profile.

15. The coated medical device of claim 14, wherein the first release profile, the second release profile, and the third release profile differ by release rate, time of release, or total amount released of the therapeutic agents.

16. A coated medical device comprising:
a tubular sidewall, wherein the sidewall comprises a plurality of struts each having an outer surface that is adapted for exposure to a body lumen, an inner surface opposite the outer surface, and at least one side surface adjacent to the outer surface and the inner surface and connecting the outer surface and the inner surface;
wherein the inner surface of at least one strut has at least a portion that is coated by a first coating composition by a first coating process, wherein the first coating composition comprises a first therapeutic agent and first polymer;
wherein the side surface of said at least one strut has at least a portion that is coated by a second coating composition by a second coating process, wherein the second composition comprises a second therapeutic agent and second polymer, and wherein the second coating composition is different from the first coating composition;
wherein the first therapeutic agent is released with first release profile and the second therapeutic agent is released with a second release profile which is different than the first release profile.

17. The coated medical device of claim 16, wherein the first release profile and the second release profile differ by release rate, time of release, or total amount released of the therapeutic agents.

18. The coated medical device of claim 16, wherein the tubular sidewall forms a stent.

19. The coated medical device of claim 16, wherein the outer surface and the side surface of said at least one strut are not coated by the first coating composition, and wherein the outer surface of the inner surface of said at least one strut are not coated by the second coating composition.

20. The coated medical device of claim 16, wherein the first therapeutic agent and the second therapeutic agent are the same.

21. The coated medical device of claim 16, wherein the first therapeutic agent and the second therapeutic agent are different.

22. The coated medical device of claim 16, wherein the first polymer and the second polymer are the same.

23. The coated medical device of claim 16, wherein the first polymer and the second polymer are different.

24. The coated medical device of claim 16, wherein the first polymer and the second polymer is biocompatible.

25. The coated medical device of claim 16, wherein the first coating composition or the second coating composition is biodegradable.

26. The coated medical device of claim 16, wherein the first coating process and the second coating process are the same.

27. The coated medical device of claim 16, wherein the first coating process and the second coating process are different.
